(19) United States
(12) Patent Application Publication
(10) Pub. No.: US 2022/0267478 A1
(43) Pub. Date: Aug. 25, 2022

(54) PEPTIDE-LOADED CARRIER SYSTEMS AND USES THEREOF

(71) Applicant: Academia Sinica, Taipei (TW)

(72) Inventors: Che-ming Jack Hu, Taipei City (TW); Chien-wei Lin, Changhua City (TW); Jung-chen Lin, New Taipei City (TW); Chen-hsueh Pai, New Taipei City (TW)

(21) Appl. No.: 17/631,698

(22) PCT Filed: Jul. 29, 2020
(86) PCT No.: PCT/US2020/043963

§ 371 Date: Jan. 31, 2022

Related U.S. Application Data
(60) Provisional application No. 62/880,421, filed on Jul. 30, 2019.

(51) Int. Cl. C07K 17/08; A61K 47/62; A61K 47/69; C07K 17/04; A61P 31/16; A61P 35/00

(52) U.S. Cl. CPC C07K 17/08; A61K 47/62; A61K 47/6937; C07K 17/04; A61P 31/16; A61P 35/00; C07K 2319/41; C07K 2319/42; C07K 2319/43

(57) ABSTRACT

A carrier system that includes a nanocarrier and a peptide non-covalently associated with the nanocarrier. The peptide contains an adaptor peptide sequence fused to the N-terminus of a target peptide, the adaptor peptide sequence being designed to facilitate the association to the nanocarrier. Also disclosed is a method for improving the immunogenicity of a peptide antigen by fusing it to an adaptor peptide sequence to form an immunizing peptide and contacting the immunizing peptide with a compatible nanocarrier. Further, a method is provided for treating a condition by immunization with a target peptide that has been fused to an adaptor peptide sequence and thereby associated with a nanocarrier. The method induces an immune response against the target peptide for treating cancer, viral infection, bacterial infection, parasitic infection, autoimmunity, or undesired immune responses to a biologies treatment.

### BACKGROUND

Personalized cancer vaccines have been developed that show promising results in animal studies and early clinical trials. Yet, these studies and trials revealed several critical challenges that need to be resolved before the potential of personalized vaccines can be fully realized. For example, stimulation of T cells against multiple cancer peptide targets, necessary for a strong anti-cancer effect, is a challenging task that demands novel technology for vaccine delivery. Current clinical trial regimens include as many as 10 booster vaccinations to elicit observable cellular immunity (see Sahin et al., Nature 547: 222-226; Keskin et al., Nature 565:234-239; Hilf et al., Nature 565:240-245; and Ott et al., Nature 547:217-221), resulting in prolonged treatment time and compromised treatment effectiveness.

Synthetic nanocarriers have been tested as delivery vehicles for peptide antigens. Such nanocarriers are thought to shield the peptide from the harsh extracellular environment following administration and to promote its cellular uptake, leading to enhanced effectiveness. In addition, immunological adjuvants have been incorporated into the nanocarrier for synchronous delivery of immuno-potentiating signals and peptides, ideal for eliciting an immune response (see Crouse, J. et al., Nature Rev. Immunol. 15:231-42). However, this approach requires complicated chemistry or use of non-biocompatible materials (see Kuai, R., et al., Nature Materials 16:489-496; Li, A. W. et al., Nature Materials 17:528-534; Luo, M., et al., Nature Nanotechnol. 12:648-654; and Liu, H., et al., Nature 507:519-522), raising both logistical and safety concerns.

The need exists to develop a carrier system in which peptides of various physicochemical characteristics can readily associate with a nanocarrier without employing laborious chemistry. This approach will facilitate multi-peptide formulation and delivery, thereby expanding the research and clinical applications of peptide-based therapeutics. In particular, a strategy to deliver varying peptide antigens without compromising their immunogenicity is needed for effective multi-antigen vaccine development. The carrier system technology is critical for effective neoantigen vaccination and is also applicable in the areas of infectious disease management and immune tolerance induction.

### SUMMARY

To efficiently deliver a target peptide as described above, a carrier system is provided that includes a nanocarrier and a peptide non-covalently associated with the nanocarrier. The peptide is made up of an adaptor peptide sequence fused to the N-terminus of the target peptide. The nanocarrier has a core which can be hydrophobic or hydrophilic. The nanocarrier also has a surface, which can have a net negative charge, a net positive charge, or one or more functional groups. The adaptor peptide sequence is designed to associate non-covalently with the hydrophobic core, the hydrophilic core, the surface having a net negative charge, the surface having a net positive charge, or the surface bearing one or more functional groups.

Also provided is a method for improving the immunogenicity of a peptide antigen. The method includes the steps of fusing the peptide antigen to an adaptor peptide sequence to form an immunizing peptide and contacting the immunizing peptide with a nanocarrier such that the immunizing peptide stably associates noncovalently with the nanocarrier. The target peptide is an MHC class I-restricted epitope or an MHC class II-restricted epitope, the nanocarrier has a hydrophilic core, and the adaptor peptide sequence includes two or more hydrophilic amino acids selected from D, E, R, K, and H.

Further disclosed is an immunization method for treating a condition in a subject. The method is carried out by fusing a target peptide to an adaptor peptide sequence to form an immunizing peptide, contacting the immunizing peptide with a nanocarrier such that the immunizing peptide stably associates noncovalently with the nanocarrier to form a carrier system, and administering the carrier system to the subject, thereby raising an immune response to the target peptide. The target peptide is an MHC class I-restricted epitope or an MHC class II-restricted epitope and the method can be used for treating a subject suffering from cancer, viral infection, bacterial infection, parasitic infection, autoimmunity, or undesired immune responses to a biologics treatment.

The details of one or more embodiments are set forth in the description and the examples below. Other features, objects, and advantages will be apparent from the detailed description, from the drawings, and also from the appended claims.

### BRIEF DESCRIPTION OF THE DRAWINGS

The description below refers to the accompanying drawings, of which:

FIG. 1 is a schematic representation of a peptide of the invention. It includes an adaptor peptide sequence (compatibility affording segment), an optional spacer segment (cleavable linker), and a target peptide. Each circle represents a single amino acid.

FIG. 2 shows schematics of different nanocarriers for use in carrier systems with the peptide shown in FIG. 1.

FIG. 3 shows exemplary carrier systems of the invention in which the peptide associates with a nanocarrier core via hydrophobic or hydrophilic interactions.

FIG. 4 shows additional carrier systems encompassed by the invention in which peptides interact with surface charges of the nanocarrier.

FIG. 5 shows a carrier system having a functional group, i.e., an antibody, on the nanocarrier surface that binds to an epitope on the peptide to a nanocarrier via an antigen-bearing adaptor;

FIG. 6 shows another example of a carrier system with a surface functional group interacting with a peptide.

FIG. 7 shows a carrier system having a self-assembly moiety on the nanocarrier surface and the same moiety fused to the target peptide.

FIG. 8 are graphs of absorbance versus retention time for HPLC analyses of hollow thin-shell nanoparticles and hydrophilic peptides A (gp100; KVPRNQDWL—SEQ ID NO: 1) and B (Trp1m; TAYRYHLL—SEQ ID NO: 2) and unmodified tyrosinase-related protein 2 (Trp2; SVYDFFVWL—SEQ ID NO: 3)(upper graph) and control Trp2 peptide in DMSO (lower graph).

FIG. 9 are graphs of absorbance versus retention time for HPLC analyses of hollow thin-shell nanoparticles encapsulating Trp2 fused at its N-terminus with peptide adaptor/spacer sequence D₃G₃ (D₃G₃-Trp2; upper graph) and control D₃G₃-Trp2 peptide in DMSO (lower graph).

FIG. 10 is a bar graph showing percentage of CD8 T cells producing interferon-gamma (IFN-γ) after challenging splenocytes with Trp2 peptide. The splenocytes were isolated from mice vaccinated with the indicated Trp2 peptides encapsulated in hollow thin-shell nanoparticles together with the stimulator of interferon genes (STING) agonist cyclic di-GMP.

FIG. 11A is a graph of tumor size versus days post-inoculation of B16F10 murine melanoma cells. Mice were vaccinated with (i) hollow thin-shell nanoparticles loaded with the modified D₃G₃-Trp2 peptide (NP), (ii) the modified D₃G₃-Trp2 peptide plus cyclic di-GMP (Peptide+dcGMP), (iii) the modified D₃G₃-Trp2 peptide plus poly(I:C) (Peptide+poly(IC)), or PBS.

FIG. 11B is a plot of survival versus days post-inoculation of B16F10 murine melanoma. Inoculations were as described in the legend to FIG. 11A.

FIG. 12 are graphs of absorbance versus retention time for HPLC analyses of hollow thin-shell nanoparticles (top graph) loaded simultaneously with three modified target peptides, i.e., D₃G₃-modified RalBP1-associated Eps domain-containing protein 1 (D₃G₃-Resp1), D₃G₃-modified ADP dependent glucokinase (D₃G₃-Adpgk), and D₄G₃-modified dolichyl-phosphate N-acetylglucosaminephosphotransferase (D₄G₃-Dpagt1); and control peptides in DMSO (bottom three graphs).

FIG. 13A is a bar graph showing percentage of IFN-γ producing CD8 T cells after challenging splenocytes with Resp1, Adpgk, and Dpagt1 peptides. The splenocytes were isolated from mice vaccinated with (i) hollow thin-shell nanoparticles loaded with the three modified peptides D₃G₃-Resp1, D₃G₃-Adpgk, and D₄G₃-Dpagt1 and STING agonist cyclic di-GMP (Nanoparticle), (ii) the three unmodified peptides plus cyclic di-GMP (Peptide+cdGMP), and (iii) the three unmodified peptides plus poly(I:C) (Peptide+poly(IC)).

FIG. 13B is a graph of tumor size versus days post-inoculation of MC38 murine colon adenocarcinoma cells into mice vaccinated as described in the legend to FIG. 13A.

FIG. 14 is a graph of absorbance versus retention time for HPLC analyses of hollow thin-shell nanoparticles containing D₃G₃-Trp2 and hydrophilic peptides C (gp100) and D (Trp1m).

FIG. 15 is a bar graph showing percentage of IFN-γ-producing CD8 T cells after challenging splenocytes with ovalbumin epitope OVA₂₅₇₋₂₆₄ peptide. The splenocytes were isolated from mice vaccinated with the indicated OVA₂₅₇₋₂₆₄ peptides encapsulated in hollow thin-shell nanoparticles together with cyclic di-GMP.

FIG. 16A includes bar graphs showing percentage of IFN-γ-producing CD8 T cells (top half) and IFN-γ-producing CD4 T cells (bottom half) after challenging splenocytes with the indicated hydrophobic unmodified peptide antigens. The splenocytes were isolated from mice vaccinated with the indicated peptides encapsulated in hollow thin-shell nanoparticles together with cyclic di-GMP.

FIG. 16B includes bar graphs showing percentage of IFN-γ-producing CD8 T cells (top half) and IFN-γ-producing CD4 T cells (bottom half) after challenging splenocytes with the indicated hydrophilic unmodified peptide antigens. The splenocytes were isolated from mice vaccinated as described in the legend for FIG. 16A.

FIG. 17 is a schematic showing a facile and unified process for manufacturing personalized cancer vaccines targeting neoepitopes.

FIG. 18A is a graph of absorbance versus retention time for HPLC analyses of hollow thin-shell nanoparticles containing 7 distinct B16 melanoma neoepitopes, designated as M05, M24, M27, M28, M30, M33 and M50 (Group I). These 7 out of 21 neoepitopes predicted using IEDB consensus method version 2.5 were arbitrarily grouped together to prepare nanoparticles.

FIG. 18B is a graph of absorbance versus retention time for HPLC analyses of hollow thin-shell nanoparticles containing 7 distinct B16 melanoma neoepitopes, designated as M08, M12, M17, M21, M25, M29, and M44 (Group II).

FIG. 18C is a graph of absorbance versus retention time for HPLC analyses of hollow thin-shell nanoparticles containing 7 distinct B16 melanoma neoepitopes, designated as M20, M22, M36, M45, M46, M47 and M48 (Group III).

FIG. 19A is a bar graph showing percentage of IFN-γ-producing CD8 T cells after challenging splenocytes with neoepitopes predicted in murine B16 melanoma. The splenocytes were isolated from mice vaccinated with the modified neopeptides encapsulated in hollow thin-shell nanoparticles together with cyclic di-GMP. The neoepitope candidates, listed in the legends to FIGS. 18A-18C, were predicted using IEDB consensus method version 2.5.

FIG. 19B is a bar graph showing percentage of IFN-γ-producing CD8 T cells after challenging splenocytes with neoepitopes predicted in murine B16 melanoma using DeepHLApan. The splenocytes were isolated as described in the legend to FIG. 19A.

FIG. 20A is a bar graph showing percentage of IFN-γ-producing CD8 T cells after challenging splenocytes with neoepitopes predicted by DeepHLApan in a colorectal cancer patient. The splenocytes were isolated from human HLA-transgenic mice vaccinated with the modified neopeptides encapsulated in hollow thin-shell nanoparticles together with cyclic di-GMP.

FIG. 20B is a bar graph showing percentage of IFN-γ-producing CD8 T cells after challenging splenocytes with neoepitopes predicted by DeepHLApan in a second colorectal cancer patient. The splenocytes were isolated as described above in the legend to FIG. 20A.

FIG. 21A is a schematic showing induction of tolerance to a peptide antigen by modifying the peptide with a peptide adaptor sequence and encapsulating it in a nanocarrier together with an immunosuppressor.

FIG. 21B is a timeline for inducing tolerance in mice to OVA₃₂₃₋₃₃₉ with D₄G₃-modified OTII nanoparticles (D₄G₃-OTII; SEQ ID NO: 4).

FIG. 22A is a plot of flow-cytometry showing percentages of CD25⁺Foxp3⁺ T_{reg} populations in splenocytes derived from a mouse inoculated with the indicated aspirin/peptide formulations or controls. NP=nanoparticle.

FIG. 22B is a bar graph showing the mean percentage of CD25⁺Foxp3⁺ T_{reg} among total CD4 T cells in mice inoculated as indicated.

FIG. 22C is a bar graph showing the total number of CD25⁺Foxp3⁺ T_{reg} cells in the mice inoculated as above.

FIG. 22D is a plot of flow-cytometry showing percentage of Foxp3⁺ T_{reg} cells among OTII-tetramer-positive CD4 T cells in splenocytes derived from a mouse inoculated as indicated.

FIG. 22E is a bar graph showing the mean percentage of Foxp3⁺ T_{reg} cells among OTII-tetramer-positive CD4 T cells from mice inoculated as shown.

FIG. 23. Schematic illustrating the nanoparticle incubation schedule and protocol for the assessment of immune tolerance induction in vitro.

FIG. 24 includes bar graphs showing the percentage of JAWSII dendritic cells expressing CD80 (upper left panel), CD86 (upper right panel), MHC I (bottom left panel) and MHC II (bottom right panel) assessed by flow cytometric analysis after the cells were co-cultured with the indicated aspirin/peptide formulations.

### DETAILED DESCRIPTION

The carrier system of the invention includes a nanocarrier and a peptide non-covalently associated with the nanocarrier.

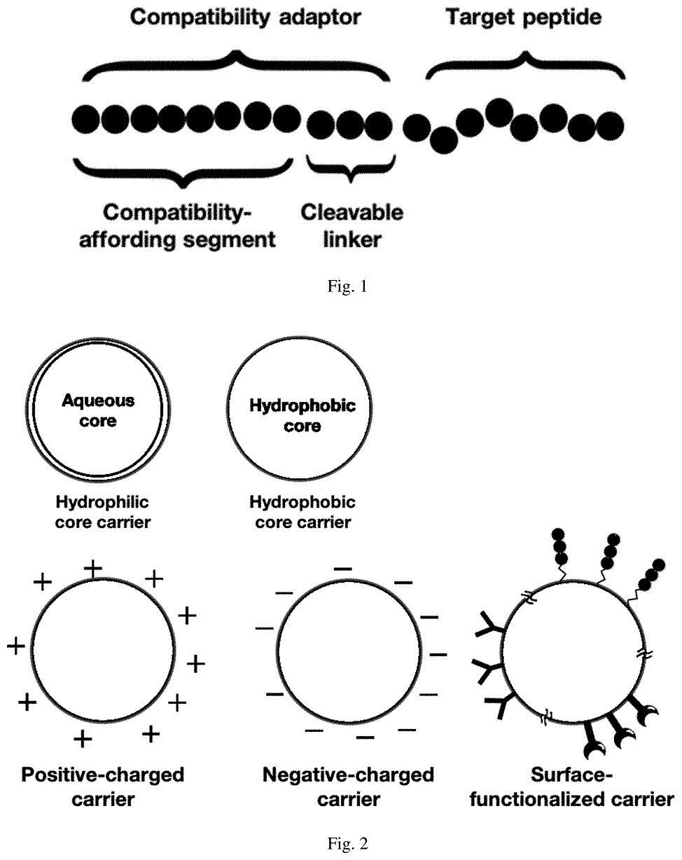

As mentioned above, the peptide contains an adaptor peptide sequence fused to the N-terminus of a target peptide. See FIG. 1.

The adaptor peptide sequence can include two or more hydrophilic amino acids selected from D, E, R, K, and H. The adaptor peptide sequence containing hydrophilic amino acids can be fused to a hydrophobic target peptide, thereby rendering the fusion peptide hydrophilic. The adaptor peptide sequence can also be fused to a hydrophilic target peptide. The sequence of the adaptor peptide sequence can be, but is not limited to, Dₙ, Eₙ, (DE)ₙ, (DX)ₙ, or (EX)ₙ, where n is an integer from 2 to 20 and X is any amino acid. In particular examples, amino acids P, A, V, I, L, M, F, Y, W are excluded from the adaptor peptide sequence set out in this paragraph.

Other adaptor peptide sequences that can be used include two or more hydrophobic amino acids selected from A, V, I, L, P, F, W, and M.

Further, adaptor peptide sequences having positively charged amino acids, e.g., K R, and H, are within the scope of the invention, as well as adaptor peptide sequences having negatively charged amino acids, e.g., D and E.

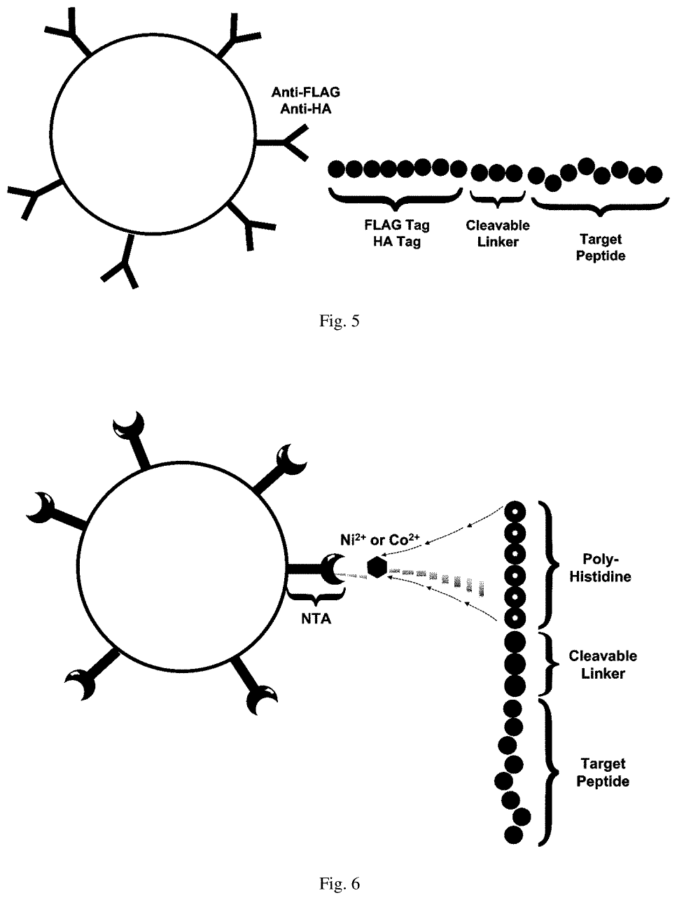

In addition, adaptor peptide sequences can be those that bind to functional groups, e.g., FLAG tag (DYKDDDK—SEQ ID NO: 5), HA tag (YPYDVPDYA—SEQ ID NO: 6), and Myc tag (EQKLISEEDL—SEQ ID NO: 7), each of which can bind to a respective anti-tag antibody. See FIG. 5.

Poly-histidine can also be included in the adaptor peptide sequence. See FIG. 6.

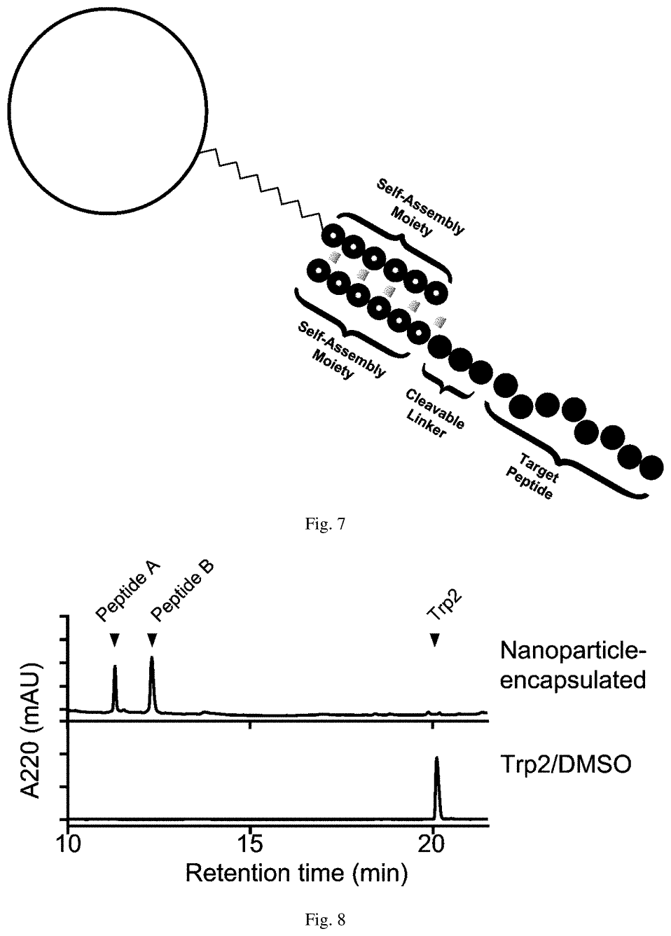

Finally, as shown in FIG. 7, the adaptor peptide sequence can be a self-assembly sequence (e.g. alpha helices, Q11 peptides, ionic-complementary self-assembling peptides, and long-chain alkylated peptides). Additional self-assembly sequences are described in Sun et al., Int. J. Nanomedicine 2017:73-86 and Li et al., Soft Matter, 15:1704-1715.

The peptide in the disclosed carrier system can include a spacer segment fused between the target peptide and the adaptor peptide sequence. The spacer segment can include two or more amino acid residues selected from G, A, S, and P. An exemplary spacer segment has the amino acid sequence Gₙ, where n is an integer from 1 to 15. The spacer segment can be susceptible to cleavage by cellular machinery such that, upon delivery of the peptide by the nanocarrier to a cell, the adaptor peptide sequence can be cleaved from the target peptide.

Specific examples of the peptide contain the adaptor peptide sequence DDD (SEQ ID NO: 8) or DDDD (SEQ ID NO: 9) and the spacer segment GGG (SEQ ID NO: 10). In this peptide, the adaptor peptide sequence is fused to the N-terminus of the spacer segment and this segment in turn is fused to the target peptide. See FIG. 1.

As mentioned above, the carrier system includes a nanocarrier. The nanocarrier can be, but is not limited to, (i) a hollow construct containing one or more aqueous cores for encapsulating hydrophilic cargoes, (ii) a solid or oil-based structure with a hydrophobic core for encapsulating hydrophobic cargoes, (iii) a carrier possessing a positive electrostatic charge for carrying negatively charged cargoes, (iv) a carrier possessing a negative electrostatic charge for carrying positively charged cargoes, and (v) a carrier having defined surface functional groups for associating with defined peptide sequences. See FIG. 2.

In a specific example, the nanocarrier is a hollow thin-shell nanoparticle having one or more aqueous core as described in Hu et al., International Application Publication 2017/165506, the content of which is incorporated herein in its entirety.

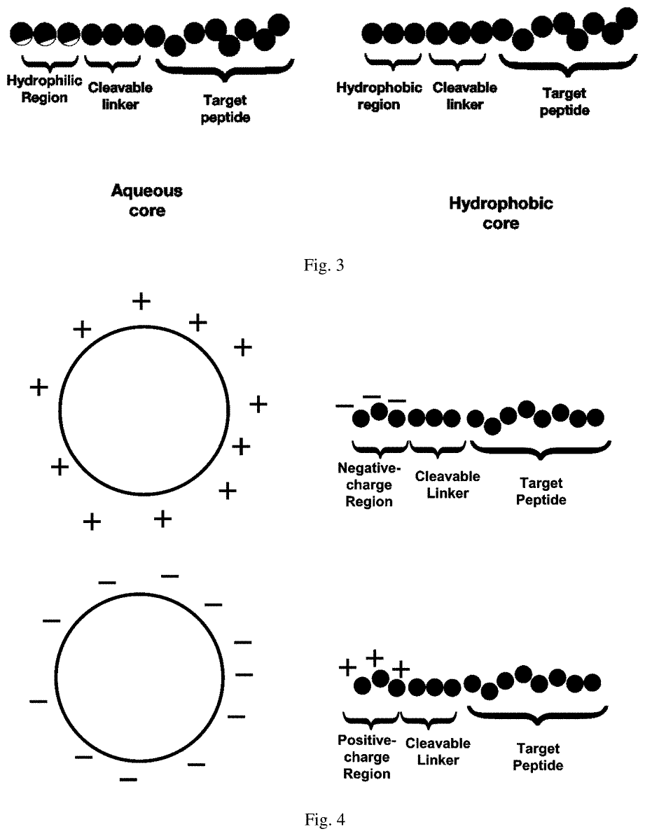

The adaptor peptide sequence described above can be selected based on the type of nanocarrier in the carrier system and the particular target peptide. For example, an adaptor peptide sequence containing hydrophilic amino acids described above can be fused to a target peptide to increase its water solubility. This water-soluble peptide can be encapsulated into the internal aqueous core of a hollow polymeric nanoparticle. See FIG. 3. Alternatively, an adaptor peptide sequence based on hydrophobic amino acids can be fused to a target peptide for incorporation into the hydrophobic compartment of a solid or oil-based carrier.

An adaptor peptide sequence containing charged amino acids can be used to facilitate the association between a target peptide and a nanocarrier bearing opposite electrostatic charges. For example, an adaptor peptide sequence containing negatively charged aspartic acids or glutamic acids can be fused to a target peptide such that the fusion peptide associates with a positively charged nanocarrier. See FIG. 4. Similarly, an adaptor peptide sequence having positively charged amino acids, e.g., lysine, arginine, and histidine, can be fused to a target peptide and thus associate with a carrier bearing a negative charge. Also see FIG. 4.

Functionalization of the carrier system can be employed to bestow the nanocarrier with a specific affinity to a particular sequence of amino acids in the adaptor peptide sequence. As mentioned above, the adaptor peptide sequences can include, e.g., FLAG tag, HA tag, and Myc tag. Target peptides fused to these adaptor peptide sequences can associate with a nanocarrier bearing on its surface antibodies that bind to the tags. See FIG. 5.

In a further example, the nanocarrier can be surface functionalized with a metal chelating agent, e.g. nitrilotriacetic acid, which has a strong affinity for poly-histidine in the presence of Ni or Co ions. An adaptor peptide sequence containing poly-histidine can be fused to a target peptide so that the fusion peptide binds non-covalently to the surface of the carrier. See FIG. 6.

Moreover, self-assembling amino acid sequences, such as alpha helices or Q11 peptides can be used as part of the adaptor peptide sequence and also for functionalizing the nanocarrier surface. With the self-assembling ability of the particular sequence, the adaptor peptide-linked target peptide can thus be coupled to the nanocarrier. See FIG. 7.

The carrier system disclosed herein can contain combinations of the nanocarriers and adaptor peptide sequence-target peptide fusions set forth, supra. For example, an exemplary carrier system includes a nanocarrier having a hydrophilic core loaded with two distinct peptides, each of which includes an adaptor peptide sequence having hydrophilic amino acids.

The carrier system can be used to deliver any desired target peptide that has been fused to an adaptor peptide sequence. In one example, the target peptide is a therapeutic peptide. In another example, the nanocarrier can be detected in vivo and the target peptide serves to localize the nanocarrier to a particular anatomical site.

Additionally, the target peptide can be an MHC class I-restricted epitope or an MHC class II-restricted epitope. Such a target peptide is used with the carrier system to enhance T cell responses to the epitope.

In particular examples, the target peptide is a cancer neo-antigen, a cancer antigen that is not a neo-antigen, a bacterial antigen, a viral antigen, or a parasite antigen.

Particular examples of target peptides include Mycobacterium tuberculosis p25, influenza nucleoprotein NP311, and cancer-associated antigens Adpgk, Dpagt, Resp1, Trp1m, and gp100. Antigenic peptides from the malaria parasite, HIV, HBV, and MERS-CoV are other examples of a target peptide.

The carrier system that includes an antigenic target peptide can also include an immunomodulator encapsulated in the nanocarrier together with the adaptor peptide sequence/target peptide fusion. The immunomodulator can be an immune response stimulator, e.g., a stimulator of interferon genes (STING) agonist, e.g., cyclic di-GMP (cdGMP), CpG-ODN, R848, and poly(I:C). Such a carrier system can be used to enhance an immune response to the target peptide.

Alternatively, the carrier system can be employed to suppress an immune response to the target peptide. In such a system, the immunomodulator encapsulated in the nanocarrier can be an immune response suppressor, for example, rapamycin, aspirin, vitamin D, a steroid, and N-acetylcysteine.

Also falling within the scope of the invention is a method for improving the immunogenicity of a peptide antigen. The method includes the steps of fusing the peptide antigen to an adaptor peptide sequence to form an immunizing peptide and contacting the immunizing peptide with a nanocarrier such that the immunizing peptide stably associates noncovalently with the nanocarrier.

Improvement of immunogenicity of a peptide antigen is assessed by comparing the immune response of the peptide antigen to the immune response of the modified peptide antigen, i.e., the immunizing peptide. The immune response is characterized by measuring the number of peptide-specific CD4+ or CD8+ T cells (“T cells”) as a percentage of total T cells, i.e., frequency. An improved immune response can therefore be defined as an increase of 1.2 to 250-fold (e.g., 1.2, 1.5, 1.8, 2, 4, 6, 8, 10, 20, 30, 40, 50, 60, 70, 80, 90, 100, 125, 150, 175, 200, 225, and 250-fold) in the frequency of peptide-specific T cells induced by the modified peptide antigen, as compared to the unmodified peptide antigen.

The target peptide is an MHC class I-restricted epitope or an MHC class II-restricted epitope, the nanocarrier has a hydrophilic core, and the adaptor peptide sequence includes two or more hydrophilic amino acids selected from D, E, R, K, and H. The target peptide antigen, adaptor peptide sequences, and nanocarriers have been described above in detail.

In a preferred embodiment, the immunizing peptide contains the adaptor peptide sequence DDD (SEQ ID NO: 8) or DDDD (SEQ ID NO: 9), a spacer segment GGG (SEQ ID NO: 10) fused to the C-terminus of the adaptor peptide sequence, and a peptide antigen fused to the C-terminus of the spacer segment.

An immunization method for treating a condition in a subject is also provided that takes advantage of the carrier system described above. The immunization method includes steps of (i) fusing a target peptide to an adaptor peptide sequence to form an immunizing peptide, (ii) contacting the immunizing peptide with a nanocarrier such that the immunizing peptide stably associates noncovalently with the nanocarrier to form a carrier system, and (iii) administering the carrier system to the subject, thereby raising an immune response to the target peptide.

In this method, the target peptide is an MHC class I-restricted epitope or an MHC class II-restricted epitope and the condition is cancer, viral infection, bacterial infection, parasitic infection, or undesired immune responses to a biologics treatment.

Without further elaboration, it is believed that one skilled in the art can, based on the disclosure herein, utilize the present disclosure to its fullest extent. The following specific examples are, therefore, to be construed as merely descriptive, and not limitative of the remainder of the disclosure in any way whatsoever. All publications cited herein are incorporated by reference in their entirety.

### EXAMPLES

### Example 1. Delivery of a Hydrophobic Peptide

A hydrophobic peptide, namely, Trp2₁₈₀₋₁₈₈ (Trp2; SVYDFFVWL—SEQ ID NO: 3), was modified by fusion to a peptide adaptor sequence and encapsulated in a nanoparticle. Trp2 is an immunodominant highly hydrophobic B16 murine melanoma epitope. This peptide was fused at its N-terminus to a hydrophilic adaptor, i.e., D₃G₃, containing three aspartic acid residues (D) as the peptide adaptor sequence and a spacer segment of three glycine residues (G) forming a cleavable linker. The peptide was synthesized by routine procedures. The sequence of the modified Trp2 peptide is DDDGGGSVYDFFVWL (D₃G₃-Trp2; SEQ ID NO: 11).

Hollow thin-shell nanoparticles having an aqueous core were prepared essentially as described in Hu et al.

To quantify peptides loaded into nanoparticles, HPLC analysis was performed as follows. Nanoparticles were lyophilized and then disrupted by adding 95% acetone. The acetone was removed by incubation at 60° C. in a dry bath, and samples were resuspended in H₂O and analyzed on an Agilent 1100 Series HPLC system using a gradient HPLC method. In an exemplary method, the starting mobile phase consisted of a 75:25 mixture of 0.1% trifluoroacetic acid in water and 0.1% trifluoroacetic acid in acetone. The second mobile phase was a 15:85 mixture of 0.1% trifluoroacetic acid in water and 0.1% trifluoroacetic acid in acetone for 20 min., followed by 10 min elution with a third phase which was 0.1% trifluoroacetic acid in acetone. Standard calibration curves for quantification of peptides were determined by absorbance at a wavelength of 220 nm.

The unmodified Trp2 peptide is poorly soluble in water, having a maximum solubility of 0.06 mM. See Vasievich, E. A., et al., Molecular pharmaceutics, 2012, 9:261-8. As such, it could not be encapsulated in the aqueous core of a hollow nanoparticle, as shown by HPLC analysis. See FIG. 8. By contrast, two hydrophilic peptides, i.e., gp100 (peptide A) and Trp1m (peptide B) were readily incorporated into the nanoparticles. See FIG. 8.

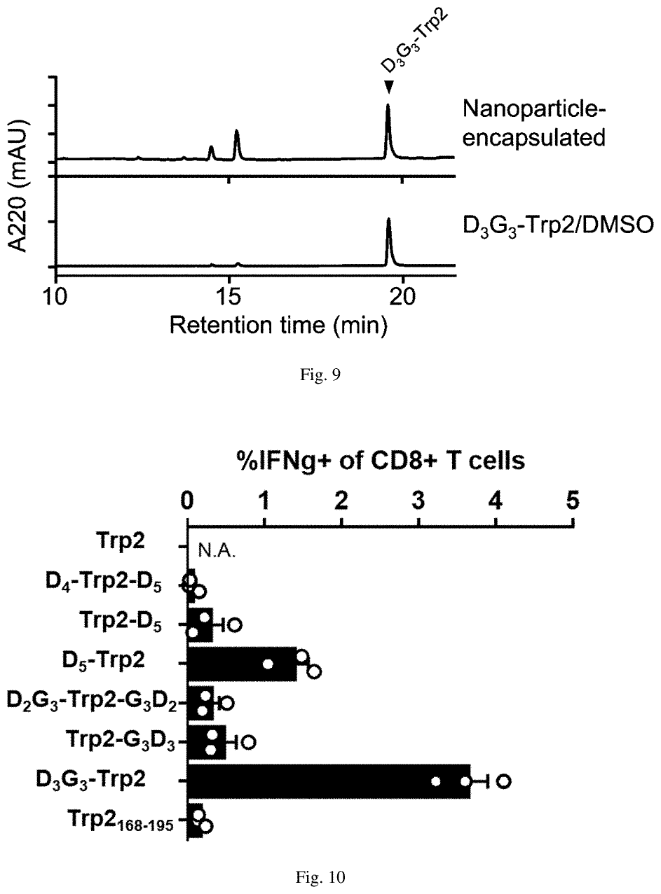

By contrast, the D₃G₃-Trp2 peptide had a solubility of >30 mM in H₂O, over 500-fold higher than the Trp2 peptide. The D₃G₃-Trp2 peptide was readily incorporated into the aqueous core of the nanoparticles. See FIG. 9.

### Example 2. Immunogenicity of Modified Peptides

The immunogenicity of modified Trp2 peptides was tested by encapsulating them in the aqueous core of hollow thin-shell nanoparticles together with a fixed amount of stimulator of interferon genes (STING) agonist cyclic di-GMP (cdGMP) and injecting them into mice.

The modified Trp2 peptides were as follows: (i) D₄-Trp2-D₅, (ii) Trp2-D₅, (iii) D₅-Trp2, (iv) D₂G₃-Trp2-G₃D₂, (v) Trp2-G₃D₃, and (vi) D₃G₃-Trp2. As Trp2 itself cannot be incorporated into the aqueous core due to its hydrophobicity, a longer Trp2 peptide, namely, Trp2₁₆₈₋₁₉₅ was encapsulated as a positive control.

Nanoparticles each containing an equivalent dose of one of the Trp2 peptides and cdGMP were prepared as described in Example 1 above and administered to C57BL/6 mice on day 0 and day 21 by subcutaneous injection at the base of the tail. On day 28, splenocytes of the vaccinated mice were isolated and examined for Trp2-specific CD8 T cell immune responses. Briefly, splenocytes from each mouse were challenged with Trp2 peptide and expression of IFN-γ in CD8 T cells was measured by intracellular cytokine staining and flow cytometry. The results are shown in FIG. 10.

All of the Trp2 peptides modified with hydrophilic aspartic acid sequences showed significant improvement in their aqueous solubilities, i.e., at least 30 mM, as compared to Trp2. Among the tested peptides, D₃G₃-Trp2, in which the hydrophilic peptide adaptor sequence together with a cleavable spacer segment fused to the N-terminus only, yielded the highest level of T cell stimulation, showing as high as 4% of CD8 T cells producing IFN-γ. See FIG. 10. It was surprising that both the inclusion of the cleavable spacer segment and the positioning of the hydrophilic adaptor and the spacer segment at the N-terminus of the target peptide were critical to obtain maximal immunogenicity of the peptide.

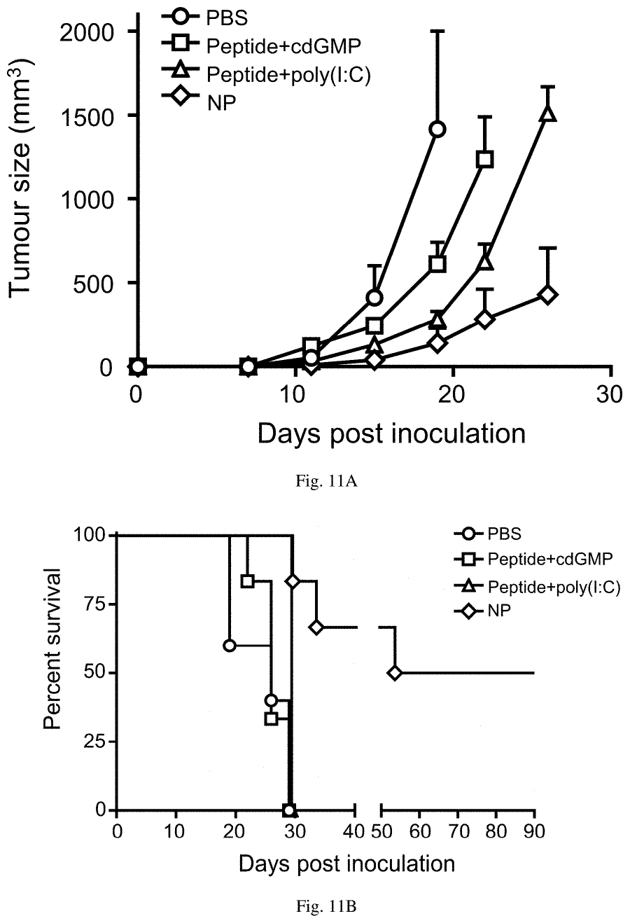

Moreover, immunizing mice with nanoparticles containing the modified D₃G₃-Trp2 led to a significant protection against B16F10 melanoma challenge. More specifically, tumor growth was inhibited (see FIG. 11A) and survival increased (see FIG. 11B) in mice immunized with these nanoparticles, showing that the D₃G₃ modification did not reduce the Trp2 peptide's anti-tumor activity.

Not to be bound by theory, it is believed that the N-terminally fused D₃G₃ peptide is readily processed by cellular proteolytic machinery, resulting in an unhampered immune response to the peptide antigen.

Furthermore, the immunogenicity of Trp2₁₆₈₋₁₉₅, a long peptide that contains amino acid sequences flanking the target epitope, i.e., amino acids 180-188, was also assessed for comparison with the hydrophilic adaptor modality. The water solubility of Trp2₁₆₈₋₁₉₅, is better than that of Trp2₁₈₀₋₁₈₈, which makes possible incorporation of the longer peptide into the aqueous core. Even so, the Trp2₁₆₈₋₁₉₅ peptide induced a ˜20-fold weaker CD8⁺ T cell response, as compared to D₃G₃-Trp2. See FIG. 10.

### Example 3. Vaccination with Cancer Neo-Epitopes

The modification strategy set forth above was also tested on three cancer neo-epitopes derived from Resp1, Adpgk, and Dpagt1 genes in MC38 murine colon adenocarcinoma cells. See Yadav, M. et al., Nature 515:572-576. These three neo-epitopes each contain a high proportion of hydrophobic amino acids and are thus inherently poorly soluble in H₂O. After fusing the D₃G₃ peptide to their N-termini, the fraction of hydrophobic amino acids in the fusion peptides were reduced to less than 40%, and their solubilities all increased to above 30 mM in H₂O.

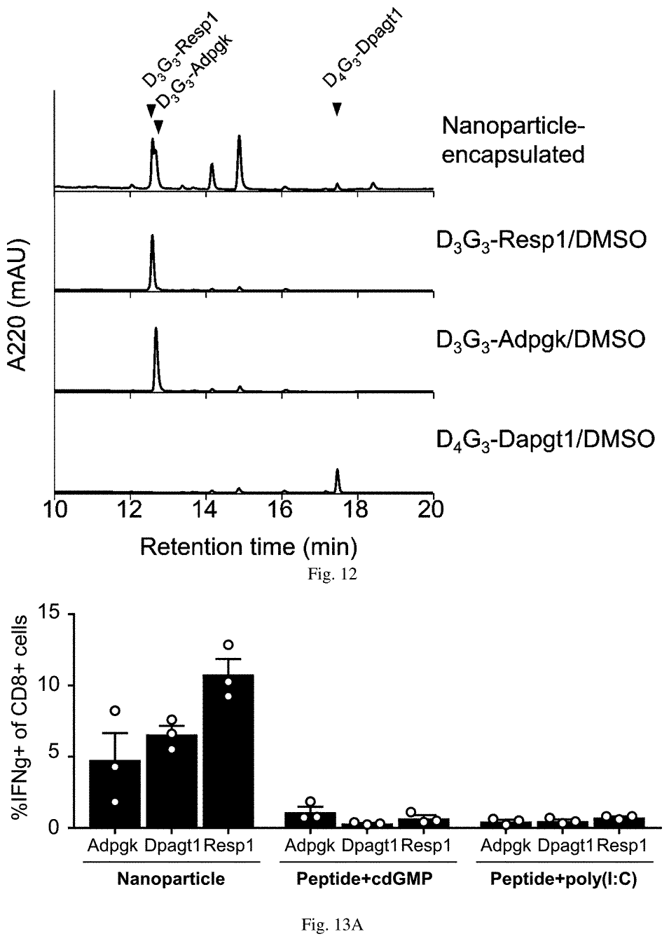

The three modified peptides, i.e., D₃G₃-Resp1, D₃G₃-Adpgk, and D₃G₃-Dpagt, were simultaneous co-encapsulated in a hollow PLGA-based nanoparticle prepared using a double emulsion process as described in Example 1. Analysis of the nanoparticles by HPLC confirmed that all three peptides were co-encapsulated. See FIG. 12.

Mice were vaccinated with (i) the nanoparticles containing the three D₃G₃-modified neo-epitope peptides and a STING agonist adjuvant, or (ii) with the unmodified neo-epitope peptides and a poly(I:C) adjuvant as set forth in Example 2. The immune responses raised by the different vaccinations were examined by CD8 T cell cytokine production and by tumor cell challenge.

The percentage of CD8 T cells producing IFN-γ was measured as described above in splenocytes challenged separately by each unmodified neo-epitope peptide. The results, show in FIG. 13A, indicated that from 5% to 12% of CD8 T cells produced IFN-γ after neo-epitope challenge. No measurable CD8 T cell response was seen in splenocytes from mice vaccinated with the free unmodified neo-epitope peptides.

Turning to tumor cell challenge, MC38 cells were injected subcutaneously into mice that had been previously immunized with (i) PBS, (ii) a mixture of the three unmodified neo-epitope peptides with poly(I:C) adjuvant, (iii) a mixture of the three unmodified neo-epitope peptides with the STING agonist cyclic di-GMP, or (iv) nanoparticles containing all three modified neo-antigen peptides and the STING agonist. The results are shown in FIG. 13B. The nanoparticle vaccination conferred significant protective immunity against subcutaneous challenge with MC38 tumor cells, as evidenced by inhibition of tumor growth. By contrast, vaccination with the three free neo-epitope peptides plus cyclic di-GMP or poly(I:C) adjuvants was significantly less effective at slowing tumor growth.

The above results make clear that a hydrophilic peptide adaptor, e.g., D₃G₃, can be employed to unify the physicochemical characteristics of a wide variety of peptides. With this strategy, it is possible to encapsulate different peptides in a hollow thin-shell nanoparticle simultaneously, irrespective of their original properties.

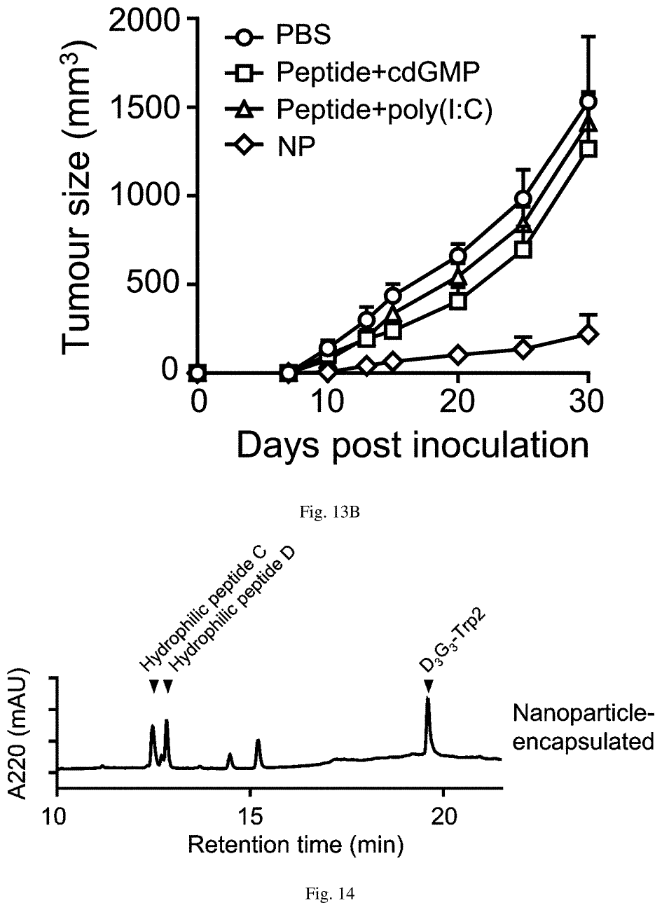

In an additional example of co-encapsulation, D₃G₃-Trp2 was successfully co-encapsulated with two hydrophilic peptides, namely, gp100 and Trp1m. See FIG. 14.

### Example 4. Modification of Water-Soluble Peptide Epitopes

The effects of hydrophilic peptide adaptors on the immunogenicity of a water-soluble peptide epitope were tested by modifying the ovalbumin peptide epitope OVA₂₅₇₋₂₆₄ (OVA; SIINFEKL—SEQ ID NO: 12) and incorporating them into nanoparticles. The modified OVA peptides were as follows: (i) D₄-OVA-D₅, (ii) OVA-D₄, (iii) D₄-OVA, (iv) D₂G₃-OVA-G₃D₂, (v) OVA-G₃D₄, and (vi) D₃G₃-OVA.

The water solubility of OVA₂₅₇₋₂₆₄ of 2 mM was improved to 50 mM by fusing to its N-terminus a hydrophilic peptide adaptor, i.e., D₃G₃. Similar solubility improvements were obtained by fusing D₃G₃ to the C-terminus of OVA₂₅₇₋₂₆₄, as well as by fusing D₄G₃ to its N-terminus or its C-terminus.

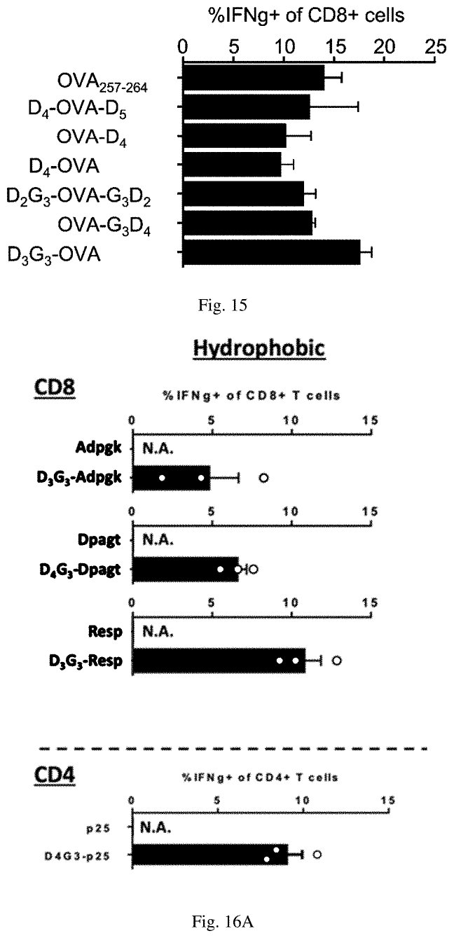

The immunogenicity of OVA and each modified OVA peptide was tested as described above. The results, shown in FIG. 15, demonstrated that OVA peptide-specific responses in splenocytes from immunized mice was increased when using the D3G3 peptide adaptor at the N-terminus, while the other modifications resulted in slightly reduced immunogenicity.

### Example 5. Modification of MHC Class I and II Epitopes

The broad applicability of the peptide adaptor modification strategy described above was examined by preparing fusion peptides as shown in Table 1 below.

TABLE 1
Modified peptide antigens

| | | | | Solubility of | |
|---|---|---|---|---|---|
| | | SEQ | | unmodified | |
| | | ID | MHC class | peptide | N-terminal |
| Peptide epitope | Amino acid sequence | NO: | restriction | epitope | mod. |
| Adpgk | ASMTNMELM | 13 | Class I | hydrophobic | D₃G₃ |
| Dpagt | SIIVFNLL | 14 | Class I | hydrophobic | D₃G₃ |
| Resp1 | AQLANDVVL | 15 | Class I | hydrophobic | D₃G₃ |
| OT-I | SIINFEKL | 12 | Class I | hydrophilic | D₄G₃ |
| OT-I | SIINFEKL | 12 | Class I | hydrophilic | D₃G₃ |
| Trp1m | TAYRYHLL | 2 | Class I | hydrophilic | D₃G₃ |
| gp100 | KVPRNQDWL | 1 | Class I | hydrophilic | D₃G₃ |
| Mycobacterium | FQDAYNAAGGHNAVF | 16 | Class II | hydrophobic | D₄G₃ |
| tuberculosis p25 | | | | | |
| OT-II | ISQAVHAAHAEINEAGR | 17 | Class II | hydrophilic | D₄G₃ |
| influenza virus | QVYSLIRPNENPAHK | 18 | Class II | hydrophilic | D₄G₃ |
| nucleoprotein | | | | | |
| NP311 | | | | | |

Each fusion peptide was loaded into the aqueous core of a hollow thin wall nanoparticle together with 1000 molecules of cdGMP as described above. All modified peptides were readily incorporated into the aqueous core of the nanoparticles, as well as the unmodified hydrophilic peptide epitopes.

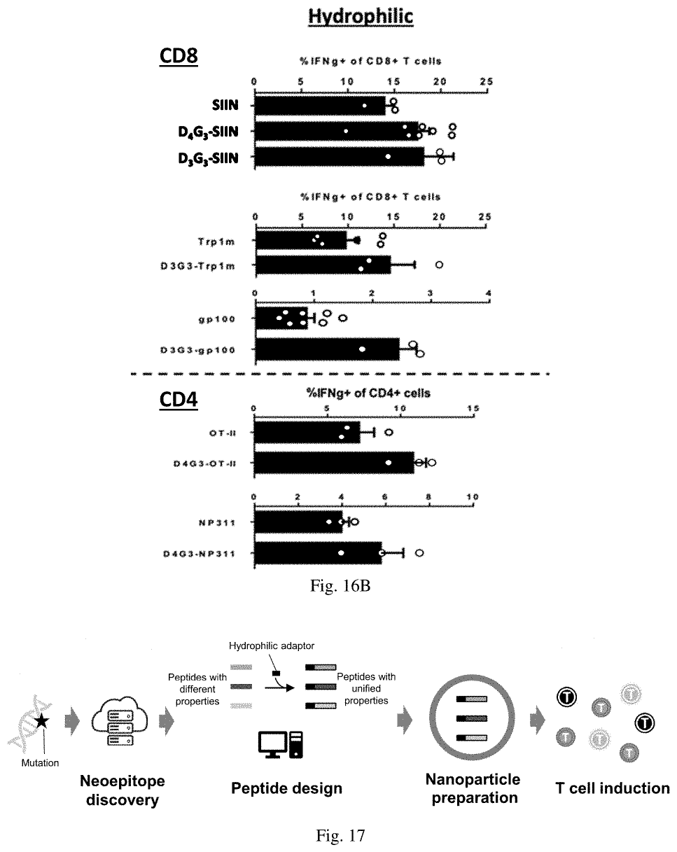

The nanoparticles were used to vaccinate mice as set forth above and T cell responses measured by intracellular cytokine staining of CD8 or CD4 T cells after challenging splenocytes isolated from vaccinated mice with the unmodified peptide epitopes. The results are shown in FIGS. 16A and 16B.

All of the modified peptide antigens tested resulted in an enhanced immune response in vaccinated mice, as compared to mice vaccinated with unmodified peptide antigens. This enhancement was shown for both CD8 and CD4 T cells, as appropriate for the tested antigen. Clearly, the peptide antigen modification strategy described above is applicable to many different peptide antigen sequences regardless of their inherent hydrophobicity and hydrophilicity.

Further, the above data shows, unexpectedly, that all of the hydrophilic peptide antigens modified with D₃G₃ or D₄G₃, have increased immunogenicity beyond the level of the corresponding unmodified hydrophilic peptide antigens, despite both being delivered by the same carrier system. See FIG. 16B. This unexpected result indicates that the peptide adaptor not only works to unify the physicochemical properties of different peptide antigens, but also improves antigen processing and presentation of the antigens.

Moreover, it bears repeating that immune responsiveness was measured in splenocytes challenged with the unmodified peptide antigens. The fact that splenocytes from mice vaccinated with modified peptide antigens responded to unmodified peptide antigens shows that the modification did not influence the specificity of T cell responses to the desired antigen sequence.

### Example 6. Identification and Immunizing of Neoantigens with Thin-Shell Nanoparticle-Encapsulated Modified Peptides

Unique mutations in individual cancer patients, known as neo-antigens, have been studied due to their potential for triggering tumor-specific immune responses. This personalized cancer vaccine approach can overcome issues such as tumor heterogeneity and patient-specific HLA haplotype differences, thereby maximizing the anti-tumor efficacy for each patient.

The peptide adaptor modifications described above are ideal for unifying the physicochemical properties of distinct peptides such as neoepitopes, thus allowing them to be co-encapsulated in thin-shell nanoparticles in a streamlined process. See FIG. 17.

The viability of this approach was tested by separately predicting two sets of 21 murine B16 melanoma neoepitopes using (i) the Immune Epitope Database (IEDB) consensus method version 2.5 and (ii) DeepHLApan software (see Wu et al., 2019, Front. Immunol. 10:2559). Individual peptides were synthesized and modified with the hydrophilic adaptor D₄G₃ and then incorporated into thin-shell nanoparticles in groups of 7 peptides.

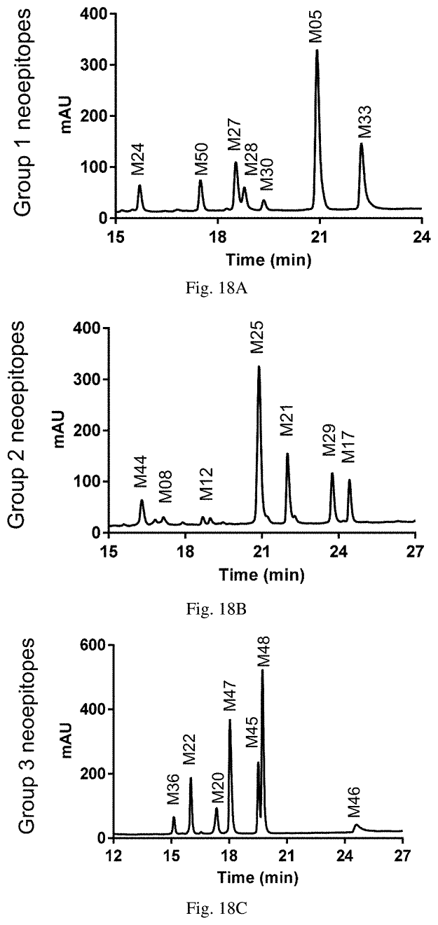

HPLC analyses showed successful encapsulation of all 21 neoepitopes predicted by the IEDB consensus method into the hollow nanoparticles in groups of 7 modified peptides. See FIGS. 18A-18C. Similar results were obtained with the 21 epitopes predicted by DeepHLApan (data not shown).

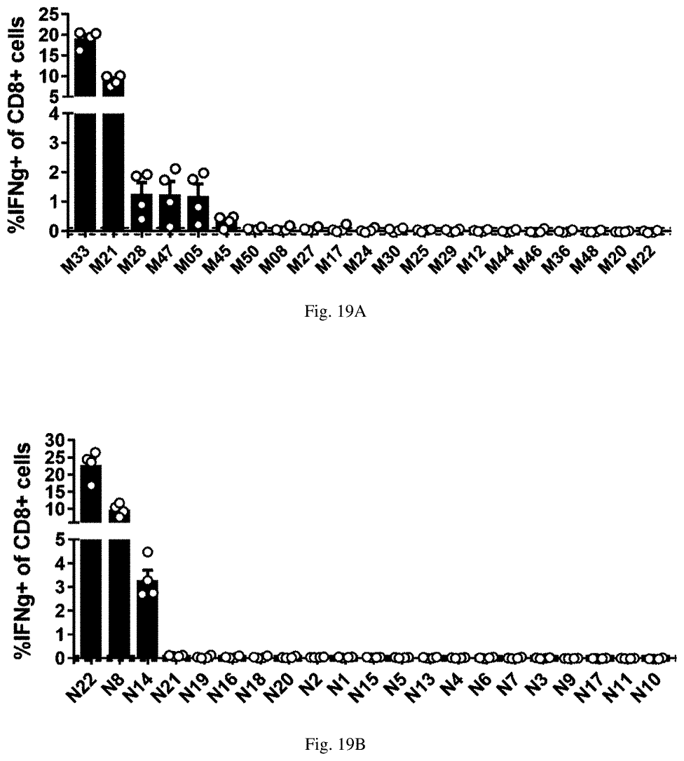

Mice were primed and boosted with the modified peptides loaded into nanoparticles with cdGMP as described above. Strong CD8+ T cell responses were detected towards 6 IEDB consensus-predicted neoepitopes (M33, M21, M28, M47, M05, and M45; see FIG. 19A) and 3 DeepHLApan-predicted neoepitopes (N22, N8, and N14; see FIG. 19B), the majority of which are novel. Among the predicted murine B16 melanoma neoepitopes, M28, M45, N22, N8 and N14 were newly discovered.

In addition, in contrast to previous literature that reported unexpected dominant CD4+ T cell responses when vaccinating mice with long synthetic peptides (see Kreiter et al., 2015, Nature 520(7549): 692-696), no significant CD4+ T cell responses were observed. Clearly, the peptide hydrophilic adaptor modification not only facilitates the manufacturing of personalized cancer vaccines, but also promotes precise neoepitope-specific immunities.

### Example 7. Identification and Immunizing of Human Cancer Neoantigens with Thin-Shell Nanoparticle-Encapsulated Modified Peptides

The peptide adaptor modification set forth above was employed on patient-derived neoepitopes. Tumor samples were collected from two colorectal cancer patients, and next-generation sequencing was performed to identify tumor-specific mutations. Sets of 9 and 21 neoepitopes were predicted using DeepHLApan, and synthesized with the hydrophilic adaptor D₃G₃ attached.

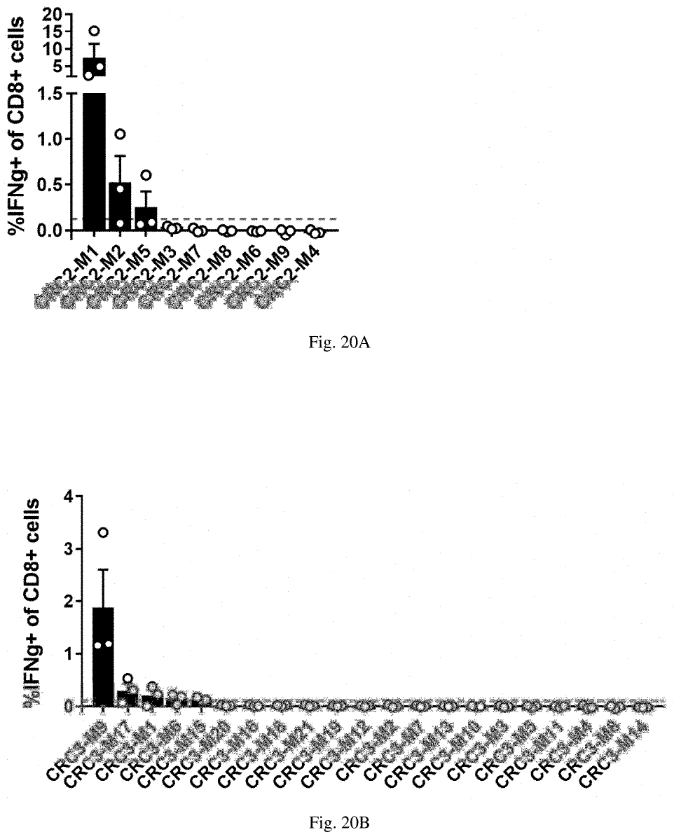

Transgenic mice bearing patient-specific HLA haplotypes were immunized with modified neoepitope-containing nanoparticle vaccines. The results showed that distinct CD8+ T cell responses were stimulated towards 3 epitopes from one patient (see FIG. 20A) and 5 epitopes from the other patient (see FIG. 20B).

These results show that the peptide adaptor design is a feasible strategy for aligning varied properties of peptides to facilitate co-delivery of neoepitopes by thin-shell nanoparticles. In addition, the identification and validation of immunogenic epitopes can be accelerated by this approach together with human HLA-transgenic mice. This offers a facile, potent platform for personalized neoantigen vaccine development.

### Example 8. Induction of Treg Cells with Modified Peptide Antigens

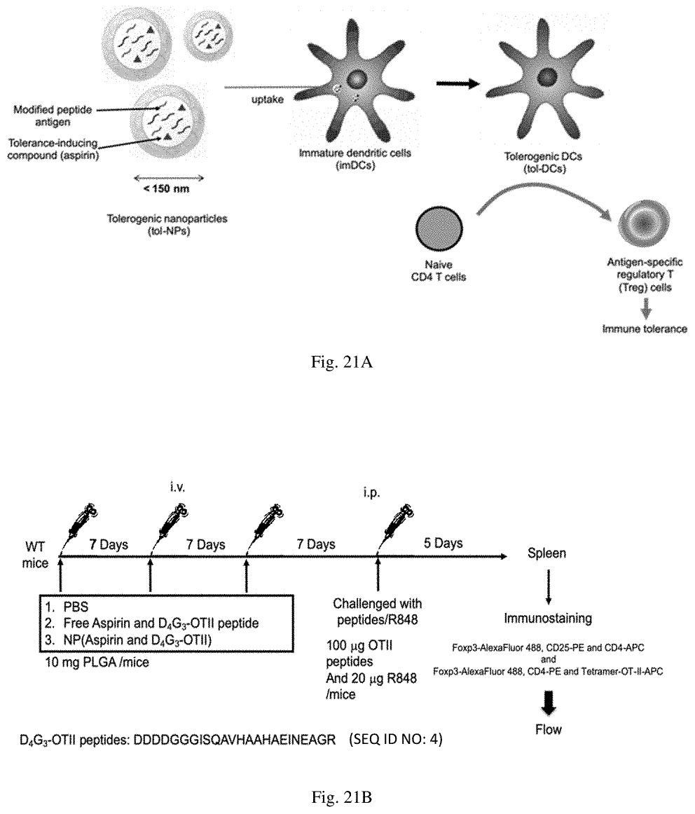

The peptide adaptor modification strategy was employed to prepare tolerance-inducing nanoparticles by co-encapsulating adaptor-modified peptide antigens with an immune suppressor, i.e., aspirin, in hollow polymeric nanoparticles. Aspirin is a compound that is capable of eliciting a tolerogenic phenotype in dendritic cells. Combining this compound with specific antigens allows for induction of antigen-specific regulatory T cells (Treg). Such Treg cells can be used for treating autoimmune diseases and for reducing immune responses to therapeutic biologics. See FIG. 21A.

Aspirin and D₄G₃-modified OTII peptide antigen (D₄G₃—OTII) were co-encapsulated in nanoparticles. The nanoparticles were used to induce tolerance as shown in FIG. 21B. Nanoparticles were injected intravenously into mice three times at one-week intervals. Seven days following the last injection, the mice were challenge with OTII peptides mixed with resiquimod, also known as R848, to simulate an immune-stimulating event. Control mice were injected with PBS or with free D₄G₃-OTII and aspirin.

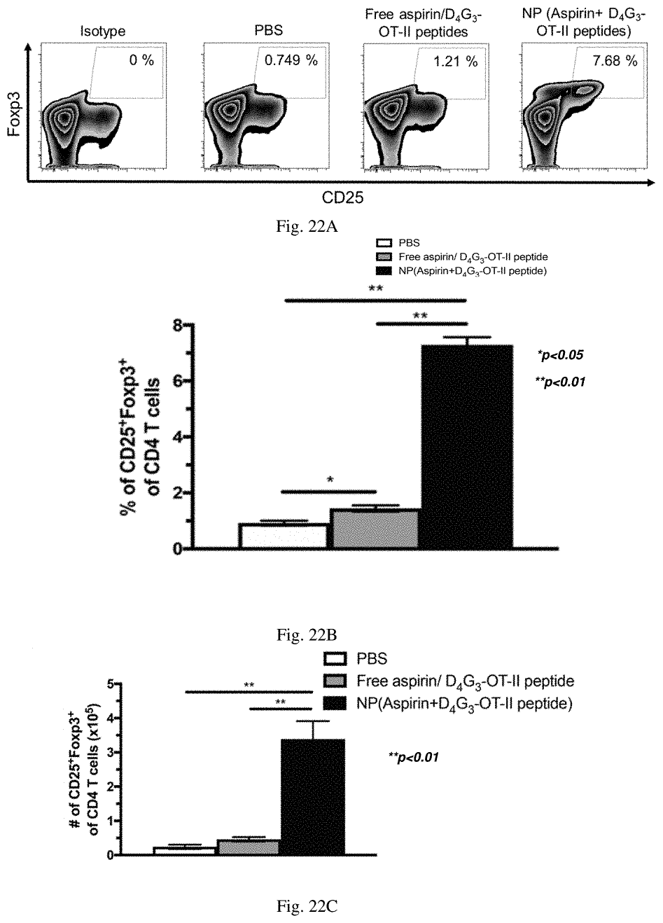

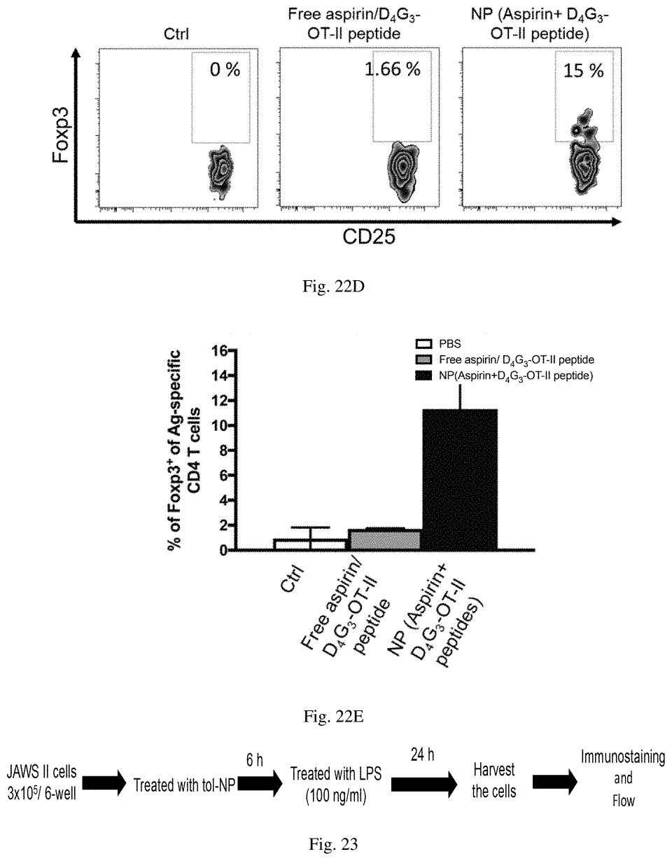

The results showed that the nanoparticle-inoculated mice produced 7 to 10-fold higher numbers of CD25⁺Foxp3⁺ Treg cells, as compared to PBS and free aspirin/peptide treated mice. See FIGS. 22A-22C.

The Treg cells produced were further analyzed by examining antigen specificity by binding to OTII tetramers. The percentage of CD4 T cells specific for OTII that were also Foxp3⁺ was as high as 15% among splenocytes isolated from mice vaccinated with D₄G₃-OTII and aspirin loaded nanoparticles, at least 9-fold higher than mice vaccinated with free D₄G₃-OTII and aspirin. See FIGS. 22D and 22E.

### Example 9. Mechanism of Tolerance Induction

Tolerogenic dendritic cells often display a phenotype with characteristically low expression of MHC molecules (e.g. MHC I and MHC II) and costimulatory molecules (e.g. CD80 and CD86) on their surface.

Surface marker expression of dendritic cells was examined in an in vitro system to ascertain how tolerance-inducing nanoparticles skew dendritic cells towards a tolerogenic phenotype.

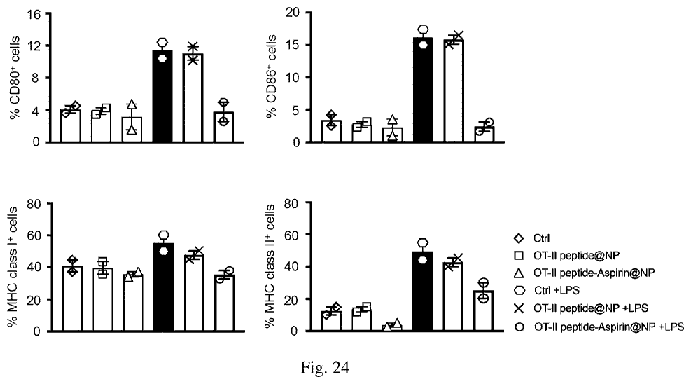

Dendritic cells were incubated with nanoparticles co-encapsulating adaptor modified OTII peptide and aspirin or adaptor-modified peptides only for 6 h, and then were stimulated with low dose of lipopolysaccharide (“LPS”). Dendritic cell phenotypes were observed 24 h later. See the experimental scheme in FIG. 23. The results are shown in FIG. 24.

As expected, LPS treatment resulted in increased expression of CD80, CD86, MHC I, and MHC II on the treated dendritic cell surfaces, as compared to vehicle control. See FIG. 24, black bars. Nanoparticles encapsulating adaptor-modified OTII peptide had little effect on LPS-induced expression of CD80, CD86, MHC I, and MHC II. See FIG. 24, fifth bar from the left in each graph. Surprisingly, nanoparticles co-encapsulating adaptor-modified OTII peptide and aspirin suppressed LPS-induced expression of CD80, CD86, MHC I, and MHC II. See FIG. 24, rightmost bar in each graph. These data show that the adaptor modification strategy can be employed to prepare tolerogenic nanoparticles to transform dendritic cells into tolerogenic dendritic cells by reducing expression of MHC molecules and costimulatory molecules on the cells.

The above results clearly demonstrate that the peptide adaptor modification strategy permits preparation of tolerogenic nanoparticles by facilitating antigen/nanocarrier coupling without compromising the epitope signature of the modified peptide.

The examples above demonstrate that the peptide adaptors can be universally applied to peptide targets to associate multiple peptide targets with a chosen carrier system. Moreover, fusion of the peptide adaptors to the target peptide unexpectedly does not reduce the immunogenicity or specificity of the target peptide. This is particularly important in the manufacturing of personalized cancer vaccines against neo-epitopes. Once tumor-specific neo-epitopes have been identified, they can be synthesized together with the peptide adaptor attached to their N-termini, without the need for detailed characterization of the epitope.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the claims.

What is claimed is:

1. A carrier system comprising a nanocarrier and a peptide non-covalently associated with the nanocarrier, the peptide containing an adaptor peptide sequence fused to the N-terminus of a target peptide, the nanocarrier having a core and a surface, wherein the core is hydrophobic or hydrophilic, the surface has a net negative charge, has a net positive charge, or bears one or more functional groups, and the adaptor peptide sequence facilitates the non-covalent association of the peptide with the nanocarrier core or surface.

2. The carrier system of claim 1, wherein the adaptor peptide sequence includes two or more hydrophilic amino acids selected from D, E, R, K, and H or the adaptor peptide sequence includes two or more hydrophobic amino acids selected from A, V, I, L, P, F, W, and M.

3. The carrier system of claim 2, wherein the core is hydrophilic and the adaptor peptide sequence is Dₙ, Eₙ, (DE)ₙ, (DX)ₙ, or (EX)ₙ, where n is an integer from 2 to 20 and X is any amino acid.

4. The carrier system of claim 3, further comprising a spacer segment fused between the target peptide and the adaptor peptide sequence, wherein the spacer includes two or more amino acid residues selected from G, A, S, and P.

5. The carrier system of claim 4, wherein the spacer segment is Gₙ, where n is an integer from 1 to 15.

6. The carrier system of claim 5, wherein the adaptor peptide sequence is DDD (SEQ ID NO: 8) or DDDD (SEQ ID NO: 9), the spacer segment is GGG (SEQ ID NO: 10), and the target peptide is fused to the C-terminus of the spacer segment.

7. The carrier system of claim 6, wherein the target peptide is an MHC class I-restricted epitope or an MHC class II-restricted epitope.

8. The carrier system of claim 7, further comprising an immune response stimulator selected from a stimulator of interferon genes (STING) agonist, CpG-ODN, R848, and poly(I:C).

9. The carrier system of claim 7, further comprising an immune response suppressor selected from rapamycin, aspirin, vitamin D, a steroid, and N-acetylcysteine.

10. The carrier system of claim 8, wherein the nanocarrier is a hollow polymeric nanoparticle.

11. The carrier system of claim 9, wherein the nanocarrier is a hollow polymeric nanoparticle.

12. A method for improving the immunogenicity of a peptide antigen, the method comprising fusing the peptide antigen to an adaptor peptide sequence to form an immunizing peptide, and contacting the immunizing peptide with a nanocarrier such that the immunizing peptide stably associates noncovalently with the nanocarrier, wherein the target peptide is an MHC class I-restricted epitope or an MHC class II-restricted epitope, the nanocarrier has a hydrophilic core, and the adaptor peptide sequence includes two or more hydrophilic amino acids selected from D, E, R, K, and H.

13. The method of claim 12, wherein the adaptor peptide sequence is Dₙ, Eₙ, (DE)ₙ, (DX)ₙ, or (EX)ₙ, where n is an integer from 2 to 20 and X is any amino acid.

14. The method of claim 13, further comprising fusing a spacer segment between the peptide antigen and the adaptor peptide sequence, wherein the spacer segment includes two or more amino acid residues selected from G, A, S, and P.

15. The method of claim 14, wherein the spacer segment is Gₙ, where n is an integer from 1 to 15.

16. The method of claim 15, wherein the adaptor peptide sequence is DDD (SEQ ID NO: 8) or DDDD (SEQ ID NO: 9), the spacer segment is GGG (SEQ ID NO: 10), and the peptide antigen is fused to the C-terminus of the spacer segment.

17. An immunization method for treating a condition in a subject, the method comprising fusing a target peptide to an adaptor peptide sequence to form an immunizing peptide, contacting the immunizing peptide with a nanocarrier such that the immunizing peptide stably associates noncovalently with the nanocarrier to form a carrier system, and administering the carrier system to the subject, thereby raising an immune response to the target peptide, wherein the target peptide is an MHC class I-restricted epitope or an MHC class II-restricted epitope and the condition is cancer, viral infection, bacterial infection, parasitic infection, autoimmunity, or undesired immune responses to a biologics treatment.

18. The method of claim 17, wherein the immunizing peptide further includes a spacer segment that is fused between the C-terminus of the adaptor peptide sequence and the N-terminus of the target peptide.

19. The method of claim 18, wherein the adaptor peptide sequence is DDD (SEQ ID NO: 8) or DDDD (SEQ ID NO: 9) and the spacer segment is GGG (SEQ ID NO: 10).

20. The method of claim 17, further comprising incorporating into the nanocarrier an immune response stimulator selected from a stimulator of interferon genes (STING) agonist, CpG-ODN, R848, and poly(I:C), wherein the target peptide is a cancer antigen, a viral antigen, a bacterial antigen, or a parasite antigen.

21. The method of claim 17, further comprising incorporating into the nanocarrier an immune response suppressor selected from rapamycin, aspirin, vitamin D, a steroid, and N-acetylcysteine, wherein the target peptide is an autoantigen and the administering the carrier system induces tolerance to the autoantigen.

22. The method of claim 20, wherein the nanocarrier is a hollow polymeric nanoparticle.

23. The method of claim 21, wherein the nanocarrier is a hollow polymeric nanoparticle.


SEQUENCE LISTING (18 sequences)

SEQ ID NO: 1 - protein, 9 aa, Artificial Sequence
  synthetic peptide
KVPRNQDWL

SEQ ID NO: 2 - protein, 8 aa, Artificial Sequence
  Synthetic peptide
TAYRYHLL

SEQ ID NO: 3 - protein, 9 aa, Artificial Sequence
  Synthetic peptide
SVYDFFVWL

SEQ ID NO: 4 - protein, 24 aa, Artificial Sequence
  Synthetic peptide
DDDDGGGISQAVHAAHAEINEAGR

SEQ ID NO: 5 - protein, 7 aa, Artificial Sequence
  Synthetic peptide
DYKDDDK

SEQ ID NO: 6 - protein, 9 aa, Artificial Sequence
  Synthetic peptide
YPYDVPDYA

SEQ ID NO: 7 - protein, 10 aa, Artificial Sequence
  Synthetic peptide
EQKLISEEDL

SEQ ID NO: 8 - protein, 3 aa, Artificial Sequence
  Synthetic peptide
DDD

SEQ ID NO: 9 - protein, 4 aa, Artificial Sequence
  synthetic peptide
DDDD

SEQ ID NO: 10 - protein, 3 aa, Artificial Sequence
  Synthetic peptide
GGG

SEQ ID NO: 11 - protein, 15 aa, Artificial Sequence
  Synthetic peptide
DDDGGGSVYDFFVWL

SEQ ID NO: 12 - protein, 8 aa, Artificial Sequence
  Synthetic peptide
SIINFEKL

SEQ ID NO: 13 - protein, 9 aa, Artificial Sequence
  Synthetic peptide
ASMTNMELM

SEQ ID NO: 14 - protein, 8 aa, Artificial Sequence
  Synthetic peptide
SIIVFNLL

SEQ ID NO: 15 - protein, 9 aa, Artificial Sequence
  Synthetic peptide
AQLANDVVL

SEQ ID NO: 16 - protein, 15 aa, Artificial Sequence
  Synthetic peptide
FQDAYNAAGGHNAVF

SEQ ID NO: 17 - protein, 17 aa, Artificial Sequence
  Synthetic peptide
ISQAVHAAHAEINEAGR

SEQ ID NO: 18 - protein, 15 aa, Artificial Sequence
  Synthetic peptide
QVYSLIRPNENPAHK